(19) United States
(12) Patent Application Publication
(10) Pub. No.: US 2025/0285753 A1
(43) Pub. Date: Sep. 11, 2025

(54) METATRON REMOTE

(71) Applicant: Nikola Anastasov, Novi Sad (RS)

(72) Inventor: Nikola Anastasov, Novi Sad (RS)

(21) Appl. No.: 18/542,733

(22) Filed: Mar. 6, 2024

(51) Int. Cl. G16H 40/67; G16H 10/60; G16H 50/20

(52) U.S. Cl. CPC G16H 40/67; G16H 10/60; G16H 50/20

(57) ABSTRACT

The METATRON REMOTE is an upgrade for the Biofeedback diagnostic system that, enables it to determine the pathological condition of a patient from any distance, i.e., when the patient is not present. The Biofeedback diagnostic system utilizes a biofeedback signal between the operator, the device, and the patient, who wears a triggering sensor on the head. When the patient is not present, the METATRON REMOTE is used as part of the biofeedback loop with the device and the operator. The METATRON REMOTE is a scalar receiving-transmitting apparatus that takes information codes, i.e., signals from the Biofeedback diagnostic system, and transmits and/or receives scalar waves to the patient within a scalar quantum field.

### FIELD OF INVENTION

The METATRON REMOTE is a scalar receiving-transmitting apparatus that enables the Biofeedback diagnostic system (U.S. Pat. No. 6,549,805) to determine the pathological condition of a patient from any distance, i.e., when the patient is not present.

### BACKGROUND

The quantum approach is based on the premise that every living thing emits energy-quanta. A quantum is the smallest energy block released from every living cell in the body at the subatomic particle level.

In the quantum universe, time and space do not exist, only a scalar quantum field where the information field of all living things emits a signature frequency. One of the most essential sources of these photon emissions in living things is DNA.

Scalar waves are a phenomenon well-known to scientists. In physics, scalar waves describe electromagnetic radiation that travels through space directionlessly without decaying over time or distance.

DNA, consisting of two linked strands that wind around each other in a double helix shape, exemplifies scalar energy. Its uniqueness lies in being a carrier wave that can transmit frequencies and penetrate solid objects.

Scalar energy, or subtle energy frequencies, is generated naturally but can also be artificially produced. This energy can carry information, and scalar waves, being omnipresent, can assist in creating high-density, superconductive coherent energy fields infused with restorative information.

### DETAILED DESCRIPTION

The first time scalar was described in recorded history was by the Scottish scientist James Clerk Maxwell in the mid-1800s. Nikola Tesla, a Serbian-American inventor of radio and alternating current (AC), developed transmitters to move scalar energy back and forth, bypassing time and space without the need for wires, as it would simply materialize from one place to another through hyperspace (1904).

It is known that scalar waves can travel over great distances and can be detected and translated at very low power levels. In addition, a scalar wave can penetrate walls and barriers much better than waves in Hertzian wave-based systems. One of the most exciting applications of scalar waves is in quantum communication, where they can be used to transfer information securely and directly between different points in the universe.

U.S. Pat. No. 645,576 to Nikola Tesla describes a system for transmitting electrical energy. According to Tesla, if a system is designed and tuned properly, the transmission utilizes non-Hertzian waves (i.e., waves having non-electromagnetic properties), conventionally known as scalar waves.

Systems, apparatuses, and methods for generating and/or utilizing scalar waves are described by Lee M. Hively in the U.S. Pat. No. 9,306,527. He demonstrated that scalar waves may be transmitted and/or received.

U.S. Pat. No. 5,845,220 by Harold E. Puthoff relates to information that changes as a function of time and is communicated from a transmitting site to a receiving site by transmitting a signal comprising scalar and vector potentials without including any electromagnetic field.

Pat. No. WO 2017/026889 by Dhyan VIMAL demonstrates a device for generating scalar energy and method thereof.

Pat. No. WO 2013/155580 by Mendes Waechter describes an apparatus for generating scalar energy for domestic use, provided with an electronic circuit capable of generating an oscillating wave at a fixed frequency of 12 hertz, radiated by means of a transmitting antenna.

A biofeedback diagnostic system is described by Vladimir Nesterov in the U.S. Pat. No. 6,549,805 and includes the application of a biofeedback signal to determine a pathological condition of the patient.

By inputting into the diagnostic system some personal details of a patient which carry a signature frequency, such as name, date of birth, and address, the apparatus picks up the patient's frequency. Fundamentally, the apparatus finds the patient's frequency with some of the patient's personal details that emit the signature frequency to be picked up anywhere in the world.

If there are not enough patient details that carry the frequency, DNA from a hair or nail sample, or DNA from a pet hair sample, can be put into the METATRON REMOTE apparatus. It has already been mentioned that DNA is an example of scalar energy and carries the patient's frequency.

The transmission of the frequency patterns is received by the client's subtle

I claim:

1. METATRON REMOTE connects and takes the necessary signals from the Biofeedback diagnostic system so that it generates scalar waves that enables the system to determine a pathological condition of the patient at any distance, i.e. patient not present.

2. Just entering into the system some personal details of a patient which carry signature frequency, such as name, date of birth, address, is enough for the system to determine a pathological condition of the patient who is not present and is located at any distance. If there are not enough patient details, DNA from a hair or nail sample or DNA from a pet hair sample can be put into the METATRON REMOTE apparatus.

3. All functions and capabilities of the Biofeedback diagnostic system are fully available in remote mode, ie. when the patient is not present and METATRON REMOTE is used as part of the biofeedback loop with the device and the operator. Also, the METATRON REMOTE upgrade does not disturb the Biofeedback diagnostic system so it can be used with the same features in two ways: in local mode with sensors on the patient's head or in remote mode when the patient is not present.
